(19) United States
(12) Patent Application Publication
(10) Pub. No.: US 2021/0170193 A1
(43) Pub. Date: Jun. 10, 2021

(54) DEVICE FOR STIMULATING THE ENDOGENOUS PRODUCTION OF MELATONIN AND PROCESS FOR OPTIMIZING STIMULATION THEREOF

(71) Applicant: BEON SOLUTIONS S.R.L., Zero Branco (IT)

(72) Inventors: Alessandro FLORIAN, ZERO BRANCO (IT); Luca AZZALINI, Zero Branco (IT); Roberto BOTTOS, Zero Branco (IT); Luca MORO, Zero Branco (IT)

(73) Assignee: BEON SOLUTIONS S.R.L., Zero Branco (IT)

(21) Appl. No.: 16/760,568

(22) PCT Filed: Nov. 1, 2018
(86) PCT No.: PCT/IB2018/058582

§ 371 Date: Apr. 30, 2020

(30) Foreign Application Priority Data
Nov. 2, 2017 (IT) 102017000124940

(51) Int. Cl. A61N 5/06

(52) U.S. Cl. CPC A61N 5/0618; A61M 2021/0022

(57) ABSTRACT

A stimulation device is provided, which provides light and sound stimuli capable of inducing the production of melatonin in a human individual subjected to said stimuli. Light and sound stimuli are produced by a light source and a sound emitter connected to a wearable mask or to a wearable frame. The desired stimulation can be easily adjusted by means of mobile devices. A method is also provided based on the stimulation device for optimizing the expression of melatonin in a patient subjected to a periodic and customized treatment. The device and method provide a physical well-being to the individual and are useful for enhancing the performance of athletes and for treating various diseases and symptoms related to low metabolic levels of the melatonin hormone.

### TECHNICAL FIELD

The present invention relates to a device and a method for light and sound stimulation designed to increase in a human individual the endogenous production of melatonin, a hormone with unique antioxidant properties involved in several metabolic and physiological processes (e.g. circadian rhythm).

### BACKGROUND ART

It is well known that melatonin is a hormone produced mainly by the pineal gland (or epiphysis) and is involved in a very high number of metabolic processes. The production of this neurohormone is regulated by the rhythm of light stimulation on the retina which transfers the signal to the central nervous system and in particular to the suprachiasmatic nucleus, the body's main biological “clock”.

Melatonin performs both a hormonal function as well as a non-hormonal function.

The hormonal function is carried out through the interaction with the membrane receptors MT1, MT2 and MT3, with the main effect of acting as a chronobiotic molecule that regulates the circadian rhythms of the organism, in particular in relation to the sleep/wake and reproductive functions.

Due to the small size and lipophilicity, melatonin performs also a non-hormonal function and it is able to pass through cytoplasmic membranes and to overcome all body barriers such as the blood-brain barrier and the placenta. As a powerful antioxidant agent melatonin performs its non-hormonal function, in particular in the cell nucleus where it tends to accumulate, and in the vicinity of mitochondria, making more efficient processes connected to the electron transport chain. In addition, the molecule stimulates the function and production of other antioxidants such as glutathione, increasing its protective effect against oxidative stress damage.

The production of this molecule with remarkable beneficial properties is not constant during the life-cycle of a human being. In fact, it is regulated by specific metabolic pathways as well as by the senile decay of the pineal gland: the production is maximum until reaching puberty, which causes the first drop in production followed by the adult phase with a stable production up to 35-40 years, and subsequently the production declined further until the minimum age. Oxidative stress is known as one of the main factors triggering cellular aging and when it exceeds the threshold leads to the onset of degenerative or tumoral diseases, lethal pathologies or in any case diseases strongly affecting the quality of life of patients. The administration of melatonin as a supplement or a drug represents a well-known widespread practice although it has contraindications and side effects. In particular, once an individual takes melatonin from an external source, the pineal gland altogether ceases to produce the hormone internally and it reduces its size with the result that the subject must assimilate supplements based on synthetic melatonin for the rest of his life.

For this reason, alternative strategies were sought to increase this molecule within the body. A first strategy is based on the discovery that the light radiation at certain wavelengths stimulates or inhibits the production of melatonin. As a consequence, eyewear equipped with special colored lenses that filter light and favor the production of melatonin (e.g. as those described in the U.S. Pat. No. 5,274,403A) appeared on the market. However; these eyewear exploit only the light wave present in the external environment and do not emit any light radiation. Another similar solution is provided in the application U.S. Pat. No. 5,709,645, in which photostimulation with pulsed light sources is performed to independently stimulate the left and right visual field of each eye. However, this solution is limited to describing a pulsed light source and does not disclose any surprising technical effect related to a physiological change in the patient as a result of a treatment with such light source. Finally, a further solution for light stimulation of melatonin production was disclosed in the U.S. Pat. No. 7,678,140B2, but this invention does not involve the control of the effect produced by photostimulation on the production of melatonin before and after stimulation.

A second strategy is based on the evidence that also sound stimuli can induce the endogenous production of the melatonin hormone. In this case also the solutions known in the state-of-the-art do not provide useful teachings to control the endogenous production of the melatonin hormone and to optimize it on the basis to the needs and characteristics of the specific patient or individual subjected to stimulation.

In conclusion, there are still several technical issues in the state-of-the-art that limit the use of stimulating devices currently available for endogenous production of the melatonin hormone.

A first issue to be addressed is the need to enhance the endogenous production of melatonin through different stimulation channels and not only through the visual apparatus of the patience. A second serious issue concerns the extremely reduced stimulation customization capabilities of the known devices, which does not permit to maximize the endogenous production of melatonin according to the real needs of the patience.

### DISCLOSURE OF THE INVENTION

### Summary of the Invention

The present invention intends to provide a device for stimulating endogenous melatonin production in a human individual as well as a method based on said device that overcome the limitations set forth above.

Accordingly, the first and main object of the present invention is to provide a stimulator device which can best stimulate endogenous melatonin production by combining different stimulation mechanisms according to the characteristics and needs of the individual. In addition, a second important object of the present invention is to provide a stimulator device able to maximize and optimize the endogenous melatonin production according to the conditions and needs of a specific individual. These needs include the treatment of diseases and symptoms related to low metabolic levels of the melatonin hormone, as well as increasing the performance in athletes or simply the well-being of people.

A third important object of the present invention is to provide a stimulator device which can exploit the functionalities of common mobile electronic devices, such as a smartphone or a tablet, in order to simplify the construction of said stimulator. Such functionalities include, for example, the emission of light or sound pulses as well as the management of the entire stimulation process, and they are not covered by known melatonin stimulators, which simply provide connection to electronic mobile devices.

A fourth important object is to provide a stimulator device having a comfortable shape easy-to-wear for the patience.

Even further, a fifth important aim of the present invention is to provide a method, to be carried out by means of said device, for optimizing and customizing the stimulation so as to produce a maximum level of melatonin hormone according to the characteristics and necessity of a human subject.

Finally, a final object of the present invention is to provide in a simple manner said stimulator device and said method, by means of known and low-cost technologies.

Additional objects and advantages of the invention will be set forth in part in the detailed description which follows and in part will be obvious from the description, or may be learned by practice of the invention.

These and further objects are achieved by a stimulator device, by a stimulating process based on said device and by the use of said device and method, as set forth in the independent claims 1, 9, 12 and 13. Preferred embodiments of the invention are described in the corresponding dependent claims. The aforementioned claims, to which reference should be made for the sake of brevity, are hereinafter specifically and concretely defined and are intended as an integral part of the present description.

This device, compared to the known solutions, is characterized by the fact of combining the light and sound stimulation in order to induce the endogenous production of melatonin. Furthermore, this device, in association with a specific procedure developed by the present inventor, can be calibrated on the basis of a personalized analysis of the conditions of the specific individual, can optimize the type of stimulation (luminous or sound) and the stimulation parameters (e.g. frequency, intensity and duty-cycle) in order to maximize the efficacy in the endogenous production of melatonin. Consequently, these features yield benefits to the organism in terms of more effective regulation of its biological clock and prevention of diseases related to the accumulation of damages from oxidative stress.

In the preferred embodiment, the device according to the invention is in the form of a mask that, thanks to useful technical solutions, is not affected by the interference of stray light and environmental light and then that allows to house a smart-phone (or other mobile electronic device) in front of the user's eyes. In this way, it is not only possible to manage the entire stimulation process by means of a special app installed in the mobile electronic device, but advantageously it is possible to use the screen or the flashlight on a smartphone as a light source for photostimulation and the sound player as a generator of sound pulses for phonostimulation. Finally, the stimulator device, again through the smart-phone, is connected to an external computer in which a database containing the stimulation parameters for a plurality of subjects in different treatment conditions is stored. This allows for customization of the patience stimulation according to a self-adaptive logic.

### DESCRIPTION OF DRAWINGS

Further characteristics and advantages of the invention will become apparent from the accompanying drawings, in which:

FIG. 1 schematically depicts the stimulator device according to the present invention;

FIG. 2 represents the device according to the preferred embodiment of the present invention, wherein photo stimulators or phono stimulators have been replaced by a mobile electronic device such as a smartphone;

FIG. 3 illustrates the procedure for achieving an optimal and customized level of melatonin expression for a specific subject by means of the stimulator device according to the invention.

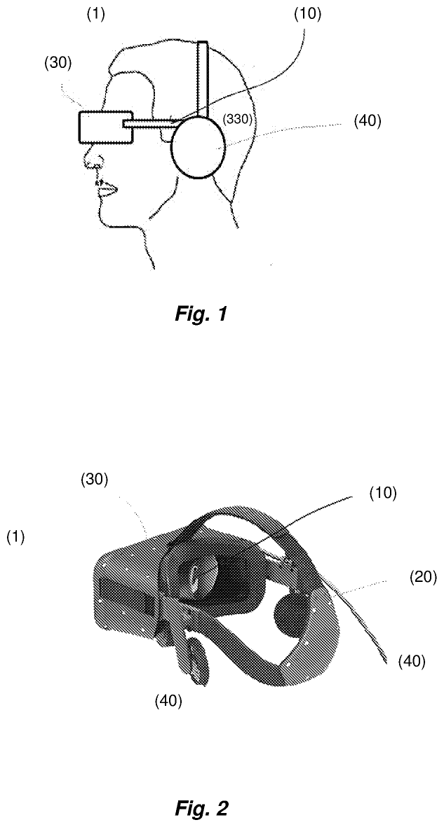

These figures illustrate and demonstrate various features and embodiments of the stimulator device and the stimulating process thereof, but are not to be construed as limiting the invention.

Description of the Stimulator Device

With reference to the aforementioned figures, the device according to the present invention, described herein by way of non-limiting example, is indicated with (1) and comprises the following elements. First, at least one stimulator for inducing endogenous production of melatonin in an human individual; such stimulator is a photostimulator device (10), a phonostimulator device (40), or, preferably, a combination thereof. The second element is a control unit (20) connected to the stimulator for managing the entire stimulation treatment so as to maximize and customize the endogenous production of melatonin. Finally, a support (30) for holding the stimulator, which preferably assumes the shape of a mask or a eyewear worn by the individual.

In association with said control unit (20), the photostimulator device (10) is able to generate light pulses having characteristics defined by parameters which include: with reference to the photostimulation as a whole, the periodicity (duty-cycle) and the duration, while with reference to the individual light pulses, the average duration, the average intensity, the average wavelength, the amplitude, the waveform and the polarization. It should be noted that the periodicity is a parameter that indicates the number of pulses generated in the unit of time by the stimulator device and it is often named “duty-cycle”. For example, a periodicity of 50 Hz means that the stimulator device generates 50 light and/r sound pulses every second. Preferably, the periodicity has a mean frequency in the range between 3 and 8000 Hz; the duration depends on the condition of the individual and varies between 0.1 and 30 minutes; the average intensity is between 1 and 300 micro Watt; also the color of the luminous pulse depends on the conditions and the sensitivity of the individual (e.g. the individual may affected by color blindness), but typically it is red (corresponding to an average wavelength of 650 nm), even if the photostimulation can be carried out with white light, or through a succession of single monochromatic impulses according to a predefined sequence (e.g. red, green and yellow).

By way of non-limiting example of the present invention, the photostimulator (10) may be a LED or a light source of another type, for example the one described in the U.S. Pat. No. 5,709,645 patent or light sources available on a smartphone as it will be described below with reference to the preferred embodiment. Between said photostimulation device (10) and the eyes of the individual, optical components can be interposed such as lenses, beam-splitters for collimating and/or subdividing the light beam, or filters to modify the wavelength and/or polarization of the light emissions. For example, a band-pass filter capable of transmitting light radiation having wavelengths between 350 and 700 nm can be interposed.

The photostimulator device (10) is conveniently placed on said support (30) in front of the eyes of the individual subjected to a photostimulation treatment so that the light pulses can stimulate the retina and therefore, through the optic nerve and other dedicated structures, the pineal gland.

For similar purposes, the phonostimulator device (40) is able to generate sound pulses having characteristics defined by parameters that include: with reference to the overall phonostimulation, the periodicity and the duration, while with reference to the individual sound pulses, the average duration, the average intensity, the average sound frequency, the amplitude, the height and the waveform/timbre. Preferably, the periodicity has a mean frequency in the range between 3 and 300 Hz; the duration depends on the condition of the individual and varies between 0.1 and 30 minutes; the average intensity is between 10 and 80 db; the frequency of the sound impulse is between 3 and 8000 Hz and depends on the conditions and sensitivity of the individual. Similarly to photostimulation, the phonostimulation can be also carried out by means of a series of sound pulses which differs for one or more characteristics according to a predefined sequence.

By way of a non-limiting example of the present invention, the phonostimulator (40) may be of the type described in the U.S. Pat. No. 4,335,710 patent, but may also be a generator of sound waves or other mechanical waves such as a piezoelectric haptic actuator, a haptic actuator of the ERM type (Eccentric Rotating Mass), a haptic actuator of the LRA type (Linear Resonant Actuators), an organic or inorganic piezoelectric transducer, a vibrating motor or a vibrotactile device. Advantageously, said phonostimulator (40) can also be the vibrating device of a smartphone, as will be hereinafter described with reference to the preferred embodiment. The phonostimulator device (10) is conveniently placed on said support (30) so that the sound pulses can stimulate the inner ear, the acoustic or vestibulocochlear nerve and therefore the pineal gland in order to induce the endogenous production of the melatonin hormone. By way of non-limiting example of the present invention, the phonostimulator (40) may take the form of one or more earpieces or headphones that can be positioned in one or more regions (330) of the individual's head, preferably above or near the auricle or of the mastoid of the subject involved in the stimulation treatment according to the invention. Said earphones can be elements separated from the support (30) of the stimulator (1) or they can be advantageously integrated with said support (30), especially when this takes the form of a mask. However other solutions are possible depending on the needs.

The set of parameters that define the characteristics of the light and sound stimulation used to stimulate the endogenous production of melatonin in an individual are named, respectively, photostimulation parameters and phonostimulation parameters and are defined by a vector called Mel-PAR, whose structure will be defined later. According to a method which will be described below, the parameters are stored, set and adjusted during the treatment by the control unit (20) with the aim of inducing the production of a maximum level of the melatonin hormone according to the characteristics and needs of a specific individual. Said control unit (20) is connected to the photostimulator device (10) and, if present, to the phonostimulator device (40) and comprises the following elements: a processor (P), a memory unit (M) and a software (S) including a set of instruction for controlling said processor (P) and said memory unit (M). The processor (P) and the memory unit (M) can be integrated into the support (30) or can be physically separated. For example, they can be the processor and the hard disk of an external computer. Furthermore, the processor (P) and the memory unit (M) can be integrated with each other. In said memory unit (M), it is stored the PAR set constituted by the Mel-PAR values of the photo and phonostimulation parameters, for example the stimulation parameters for a plurality of subjects in different treatment conditions. Finally, the control unit (20) is connected to an external computer in which a database containing the stimulation parameters for a plurality of subjects in different treatment conditions is stored. This allows to customize the stimulation for a specific individual according to a self-adaptive logic.

Description of the Stimulation Procedure

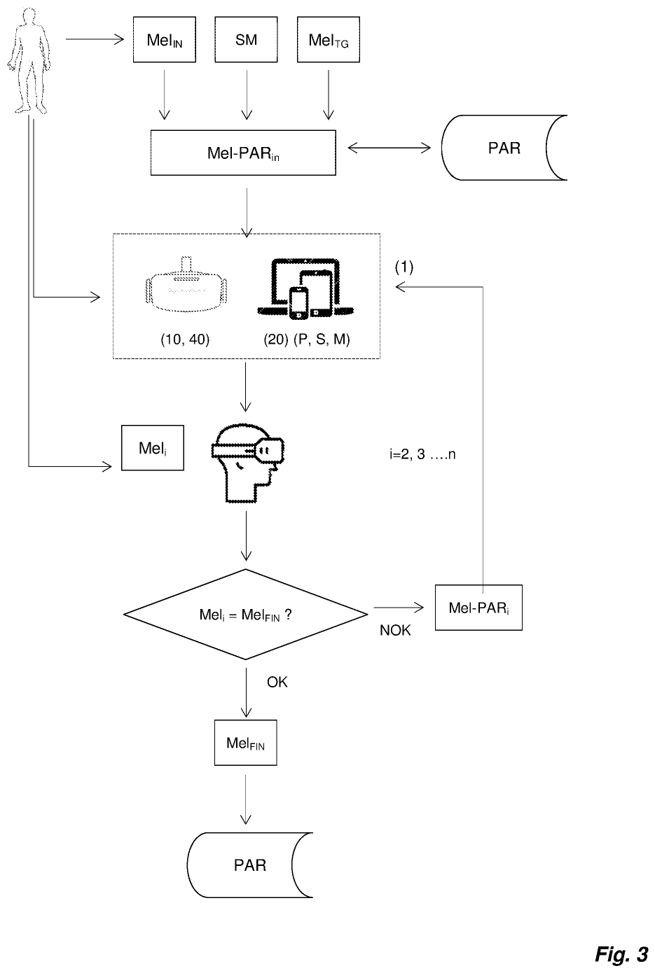

An object of the present invention also includes a method for stimulating the endogenous production of the melatonin hormone by means of the stimulation device described above. This procedure will be illustrated below by way of a non-limiting example of the present invention with reference to the enclosed FIG. 3.

In short, the procedure involves three steps: initialization of the stimulation treatment, conduction of an initial stimulation cycle, and finally optimization of the stimulation. Advantageously, said method exploits a self-adaptive logic which in the course of the treatment modifies the photo and phonostimulation conditions based on the data previously collected by the control unit (20) and stored in the memory unit (M) for the purpose of optimizing and customize endogenous melatonin production based on the conditions and real needs of a specific individual. The treatment can be performed by photostimulation i.e. with visible or infrared light pulses, or by means of phonostimulation with audible or inaudible acoustic pulses, or by using a combination of photostimulation and phonostimulation. Specifically, said stimulation method comprises the steps herein described by way of a non-limiting example of the invention.

Step 1: Initialization of the Stimulation Treatment

The procedure starts with the definition of the melatonin target value for a specific individual. This value named Mel_{TG} depends on the Mel_{IN} expression level of melatonin in said individual and on the purpose of the treatment (e.g. severe vs. mild or continuous vs. single treatment, sports performance aid vs. treatment of diseases). Typically the value of Mel_{IN}, which can be detected according to known techniques, is included in a range between 1 and 100 picograms per milliliter of saliva.

Subsequently, for each of the stimulation parameters, predefined Mel-PARᵢₙ values are selected on the basis of said Mel_{TG} and Mel_{IN} values from a PAR set of values of stimulation parameters stored in the memory unit (M) of the control unit (20) connected to the stimulator. By way of non-limiting example of the present invention, the following example table illustrates a representation of some Mel-PAR vectors contained in the PAR set (they consist of N elements, where N is a integer). It will be apparent to the skilled in the art that the Mel-PARᵢₙ vectors may have a structure different from the one provided.

| | Mel-PAR | | | | | | | | |
|---|---|---|---|---|---|---|---|---|---|
| | Patience Conditions | | | Photostimulation | | | Phonostimulation | | |
| | | Mel_{IN} | Mel₁ | Duty cycle | Duration | | Duty cycle | Duration | Frequency |
| ID | Sex | (pg/ml) | (pg/ml) | (Hz) | (min) | Colour | (Hz) | (min) | (Hz) |
| 1 | M | 10 | 28 | 0.3 | 30 | white | 0.3 | 30 | 4000 |
| 2 | F | 22 | 56 | 0.5 | 25 | white | 0.5 | 30 | 4000 |
| 3 | M | 8 | 17 | 0.5 | 15 | white | 0.5 | 15 | 4000 |
| . . . | . . . | . . . | . . . | . . . | . . . | . . . | . . . | . . . | . . . |
| N | F | 6 | 12 | 0.7 | 20 | white | 0.7 | 0 | 0 |

For example, in the assessment of the patient's condition other parameters (such as Mel_{TG}) can be included in addition to sex, initial melatonin value Mel and final melatonin value Mel₁ at the end of the initial stimulation treatment. Photostimulation or phonostimulation can also be defined in terms of other parameters among those that most affect efficacy in endogenous melatonin production on the basis of experimental data published in the medical literature or accumulated through the use of the device and of the stimulation process according to the invention. Furthermore, the same Mel-PARᵢₙ vectors may contain different elements, such as the frequency (e.g. once a week) with which the i-th treatment was performed (where i and N are integers and 1≤i≤N) on the subject.

In practice, in this step the initial Mel-PARᵢₙ values are chosen that maximize the probability for a specific individual to reach the Me_{IT} value of melatonin starting from an initial Mel value based on the stimulation parameters which are contained in the PAR set and are stored in the memory unit (M). The software (S) through the processor (P) implements the algorithm by which the initial Mel-PARᵢₙ values are chosen on the basis of statistical criteria (for example, the most frequent stimulation parameters) or of other criteria (for example, prescription from a doctor).

Step 2: Initial Stimulation Cycle

Once the initial Mel-PARᵢₙ stimulation parameters have been identified, they become the input values for an initial stimulation cycle implemented by the software (S), and then they are transferred to said processor (P) which triggers the treatment for the individual.

In the course, or at the end, of the initial stimulation cycle, the melatonin expression level in the patience is analyzed in order to obtain a new Mel₁ value. This step ends with the storage of the Mel-PARᵢₙ values in the memory unit (M): if the Mel-PARᵢₙ vector is already contained in the PAR set, then only the frequency value will be updated; otherwise, the software (S) will store the values Mel_{TG}, Mel_{IN}, Mel₁ and the other parameters of photostimulation phonostimolation or will add the new Mel-PAR vector to the PAR set.

Step 3: Optimization of the Stimulation

If the optimal melatonin level at Mel_{TG} has not been reached at the end of the initial stimulation cycle, the patient may be subjected to additional stimulation or, more generally, to an additional number of cycles n, where n is an integer n≥2.

Similarly to the initial stimulation, it is possible to subject the individual to one or both of the stimulation modalities: photostimulation through visible or infrared light pulses, to phonostimulation by audible or inaudible acoustic pulses. In each cycle, the stimulation modes can be varied according to the needs.

The photostimulation and phonostimulation parameters are defined by a Mel-PARᵢ₋₁ vector (i is an integer with 2≤i≤n) which may be the same as that associated with the initial stimulation, or it may be different. In this case the new stimulation parameters can be defined starting from the initial Mel-PARᵢₙ parameters and from the Mel₁ value in the following way. The total duration of stimulation is varied between 0.1 and 10 minutes for each picogram for each milliliter concentration of melatonin Melᵢ (wherein i is an integer, 2≤i≤n, and n is the number of additional cycles) measured in the individual subjected to treatment.

Alternatively, or in combination with the duration of stimulation, other stimulation parameters may be varied: the average wavelength of the individual pulses of a value ranging from 1 to 100 nm for each picogram per milliliter of melatonin concentration Melᵢ; the average sound frequency of the individual pulses of a value ranging from 0.1 to 200 Hz for each picogram per milliliter of melatonin concentration Melᵢ; the light intensity of a value ranging from 0.1 to 10 microwatt for each picogram per milliliter of melatonin concentration Melᵢ; the acoustic intensity of a value ranging from 0.1 to 10 db for each picogram per milliliter of melatonin concentration Melᵢ measured in said individual. In a similar way other parameters deemed relevant may be altered.

Alternatively, the choice of the Mel-PARᵢ₋₁, stimulation parameters to be adopted for the additional treatment cycles can be carried out similarly to the previous step or by choosing from the PAR collection those values that maximize the probability of reaching an expression level of melatonin in the individual equal to Mel_{TG}, starting from an initial value equal to Mel₁ (or Mel₂, Mel₃ or in general Melᵢ with the integer between 2≤i≤n, where n is the number of additional cycles). In this case also, during or at the end of each i-th additional cycle (with 2≤i≤n), the melatonin expression level in the individual is analyzed in order to obtain an Melᵢ value. Then, in the memory unit (M) associated with said processor (P) said Melᵢ value, the new parameters of stimulation Mel-PARᵢ₋₁, the Mel_{TG} value are stored as well as other parameters defining the conditions of the individual and the possible delay between two consecutive treatments i−1 and of the additional cycles n (i integer between 2≤i≤n).

This step is repeated for a number n of times until Mel reaches a Mel_{FIN} value substantially equal to Mel_{TG} or a substantially constant value: in both cases, these values correspond to the maximum or optimal melatonin expression for the patience subjected to treatment and correspond to stimulation parameters defined by the vector Mel-PARₙ=Mel-PAR_{FIN}. The step and the stimulation procedure according to the invention is completed by adding to the PAR set and storing said Mel-PARₙ vector in the memory unit (M). If Mel-PARₙ=Mel-PAR_{FIN} is already stored in the memory unit (M), then the frequency of the vector Mel-PAR_{FIN} will be correspondingly updated.

Optionally, it is possible to subject the patient to one or more periodic stimulation cycles by setting the optimal stimulation parameters stored in the memory unit (M) in said device.

### INDUSTRIAL APPLICATION

The stimulator device and the related stimulation process described above are useful in the treatment of diseases related to oxidative stress damage or melatonin hormone deficiencies. These diseases include: vertigo, tinnitus, diabetes, alopecia, insomnia, retinitis pigmentosa, maculopathy and other ocular deterioration caused by the accumulation of extracellular protein deposits.

In a preliminary experiment, a sample of 10 individuals with a severe level of maculopathy were subjected to a cycle of treatments (120 treatments for a total of 2400 minutes) by means of the stimulation device and the method according to the present invention. A significant pathology regression in 90% of cases and a stabilization of the symptom in 100% of cases were recorded. Similar results were also obtained in a sample of individuals affected by the other of the diseases mentioned above. Furthermore, in preliminary studies, a promising and unexpected efficacy of the present invention was found in the treatment and rehabilitation of stroke-affected patients.

Finally, the stimulator device and the related method have also proved to be useful as an aid for athletes in order to obtain the best performance when needed, particularly in sport practices requiring attention and concentration such as artistic gymnastics, archery, shooting, archery, basketball and other similar sports. In this case the device according to the present invention relates the melatonin production with the features (e.g. length) of the EEG brain waves to achieve the maximum athletic performance of the athlete.

### Preferred Embodiment of the Invention

In the preferred embodiment of the present invention, illustrated here by way of non-limiting example with reference to the FIG. 2, the support (30) takes the form of a mask comprising a housing (310). The photostimulator (10) and the phonostimulator (40) are a suitable smartphone positioned in a permanently removable manner in said housing (310), while the HW/SW system of said smartphone (and in particular the processor (P) and the memory unit (M)) constitutes the control unit (20) of the stimulator, which allows to fully manage the stimulation treatment through an app (S) installed in the smartphone. Finally, the smartphone exchanges data with an external computer, preferably via a wireless connection, for example with an external computer in which a database containing the stimulation parameters for a plurality of subjects in different treatment conditions is stored.

In turn, said external computer can be connected to other stimulating devices according to the present invention or, alternatively, a plurality of external computers, each of them connected to a stimulator device according to the present invention, can be connected to each other. With such a configuration, it is possible to share the PAR set (i.e. the database containing the stimulation data) stored in each of these devices so as to facilitate and speed-up the search for the best stimulation conditions for a specific individual.

In the preferred embodiment, the light sources and the sound generator integrated in the smartphone are in practice used as a photostimulator (10) and a phonostimulator (40) managed by the app (S). Preferably said light source is the screen of the smartphone, while said sound generator is the audio player of the smartphone connected to headphones or earphones integrated to the mask support (30). However, other solutions are possible. For example, the photo stimulator (10) can be the camera flashlight built into the smartphone; also the vibration alarm used in the “silent” mode can be used to stimulate the mastoid of the individual in a way useful to the endogenous production of melatonin. In addition, the headphones or earphones can be separated from the support (30). If the smartphone does not have a vibration device, alternatively it is possible to use a pair of identical haptic vibrators that are placed on the support (30) in proximity of the auricles or the mastoid of the individual. Said haptic vibrators are connected to the smartphone in such a way that allows to modify the characteristics of the vibration and therefore to optimize the endogenous production of melatonin.

To set forth the preferred embodiment of the present invention several smartphones or mobile devices present on the market can be used provided that they can generate light radiation with wavelengths included in the range between 350 and 700 nm, with a screen refresh rate between 60 and 180 Hz (preferably equal to 120 Hz), and sound waves or mechanical vibrations with a frequency included in the range between 5 and 7000 Hz. In the preferred embodiment, illustrated here by way of a non-limiting example of the present invention, the smartphone is placed in the housing (310) so that the radiation emitted by the screen or flashlight can reach the eyes of the individual subjected to the stimulation treatment. Optionally, between the light source and the eyes of the individual a band-pass filter can be interposed, capable of transmitting light radiation having wavelengths in the range between 350 and 700 nm. For this purpose it is useful any filter known to the skilled in the art, provided it is compatible with the characteristics of the mask.

The housing (310), which is shaped by means of known techniques so as to be compatible with the dimensions of the most popular smartphones on the market, has an elongated body (320) adherent to the patient's head. This useful solution prevents external light from reaching the individual's eyes disturbing the photostimulation treatment, an issue which affects devices known to the state of the art.

From the description given herein by way of non-limiting example, it is evident to the skilled in the art how this result has been achieved through a non-trivial inventive effort and represents a significant advancement in the specific context which allows to obtain greater utility with respect to known solutions, particularly in terms of ease of use and effectiveness. In conclusion, it is apparent to those skilled in the art that the present invention fully achieved the intended aim and objects. Although the description and examples above contains many details, these should not be construed as limiting the scope of the invention but as merely providing illustrations of some of the presently preferred embodiments of this invention.

In fact, numerous variants, all of which are within the general scope of the inventive concept, will be apparent to the expert of the art from the description provided herein. Therefore, any modification of the present invention which comes within the spirit and scope of the following claims is considered part of the present invention. In particular, all the details may be replaced with other technically equivalent elements. Furthermore, the order of the process steps described above can be changed according to convenience.

In the appended claims, reference to an element in the singular is not intended to mean “one and only one” unless explicitly so stated, but rather “one or more”.

Where the characteristics mentioned in any claim are followed by reference signs, those reference signs have been included for the sole purpose of increasing the intelligibility of the claims and accordingly, such reference signs do not have any limiting effect on the interpretation of each element identified by way of example, but not limitation by such reference signs.

1. A device (1) for stimulating, in a human or animal being production of melatonin comprising:
at least one photostimulator device (10) capable of generating light pulses having a duty-cycle in the range between 3 and 300 Hz, said light pulses being associated to a set of photostimulation parameters;
a control unit (20) connected to said at least one photostimulator (10) and comprising a processor (P), a memory unit (M) and a software (S) provided in said processor (fp) and in said memory unit (M), said control unit (20) being able to set and change said photostimulation parameters to enable the production of a custom amount of melatonin for said subject;
a support (30) in the form of a mask or eyewear, said support (30) comprising at least one housing (310) for said at least one photostimulator (10), said housing (310) being configured for positioning said at least one photostimulator (10) in front of the eyes of said subject, said support (30) having an elongated body (320) adhering to the head of said subject, said elongated body (320) being configured to prevent external light from reaching the eyes of said subject.

2. The device (1) according to claim 1, further comprising:
at least one phonostimulator device (40) capable of generating sound pulses having a duty cycle in the range between 3 and 300 Hz, said sound pulses being associated to a set of phonostimulation parameters;
a control unit (20) connected to said at least one phonostimulator (40) and comprising a processor (P), a memory unit (M) and a software (S) provided in said processor (fp) and in said memory unit (M), said control unit (20) being able to set and change said phonostimulation parameters to enable the production of a custom amount of melatonin for said subject;
a support (30) in the form of a mask or eyewear, said support (30) comprising at least one housing (320) for said at least a phonostimulator (20), said housing (320) being configured for positioning said at least a phonostimulator (40) on at least one region (330) of the head of said subject, said region (330) being useful for stimulating the acoustic nerve or the vestibococlear nerve by means of said sound pulses, said region (330) being the auricle or the mastoid.

3. The device (1) according to claim 1, further comprising:
at least one phonostimulator device (40) capable of generating sound pulses having a mean frequency in the range between 3 and 8000 Hz, said sound pulses being associated to a set of phonostimulation parameters, said at least one phonostimulator (40) having the form of one or more earphones or headphones separated from said support (30), said one or more headphones or headphones being positioned on at least one region (330) of the head of said subject, said region (330) being useful to stimulate the acoustic nerve or the vestibococlear nerve by means of said sound pulses, said region (330) being the earpiece or mastoid.
a control unit (20) connected to said at least one phonostimulator (40) and comprising a processor (P), a memory unit (M) and a software (S) provided in said processor (P) and in said memory unit (M), said control unit (20) being able to set and change said phonostimulation parameters to enable the production of a custom amount of melatonin for said subject.

4. The device according to claim 1, wherein:
said photostimulation parameters include: mean duration, mean intensity, mean wavelength, amplitude, waveform and polarization of the individual light pulses; duty-cycle and duration of the photostimulation, or a combination thereof;
the phonostimulation parameters include: mean duration, mean intensity, mean sound frequency, amplitude and waveform of the individual sound pulses; duty-cycle and duration of phonostimulation, or a combination thereof.

5. The device according to claim 1, wherein:
said at least one photostimulator device (10) is a mobile phone device provided with at least one light source capable of generating radiation with wavelengths in the range from 500 to 700 nm, said mobile phone device being placed in a permanently removable manner in said at least one housing (310);
said control unit (20) wherein said processor (P) and said memory unit (M) are integrated into said mobile phone device and said software (S) is an app installed on said mobile phone device;
said mobile phone device in association with said control unit (20) being able to provide a refresh rate of said at least one light source between 40 and 80 Hz.

6. The device according to in that claim 1, wherein
said at least one phonostimulator device (40) is a mobile phone device having at least one sound generator or mechanical vibration generator having frequency within the range between 5 and 7000 Hz, said mobile phone device being placed in a permanently removable manner in said at least one housing (310);
said at least one phonostimulator (40) is in the form of: a headset or headphones connected to said mobile cellular device, a headset or headphones housed in said support (30), the vibration generator mounted in said mobile phone device (i.e. to enable the so-called “silent mode”), or a combination thereof;
said control unit (20) wherein said processor (P) and said memory unit (M) are integrated to said mobile phone device and said software (S) is an app installed on said mobile phone device.

7. The device (1) according to claim 5, wherein said mobile cellular device exchanges data with an external electronic processor in wireless mode.

8. The device according to claim 1, further comprising at least one filter interposed between said at least one photostimulator device (10) and the eyes of said subject, said filter being a bandpass filter capable of transmitting light radiation having wavelengths in the range of 500 to 700 nm.

9. A process for stimulating in a human or animal being the production of an optimal amount of melatonin by means of the device according to claim 1, the method comprising:
a) defining a Me_{ITG} Melatonin Target value for a given human or animal subject, said value being in a range between 1 and 100 picograms per milliliter of saliva;
b) profiling said subject and measuring the melatonin level in said subject so as to obtain a Mel_{IN} value;
c) choosing a stimulation mode between: photostimulation by visible or invisible light pulses, phonostimulation by audible or non-audible acoustic pulses, or a combination thereof;
d) selecting predefined Mel-PARᵢₙ values for each of the stimulation parameters according to the mode selected in the preceding step, said predefined values being selected according to said Mel_{TG} and Mel_{IN} values from a set PAR of stimulation parameters values stored in said memory unit (M) associated with said processor (P), said stimulation parameters comprising: mean duration, mean intensity, mean wavelength of single pulses, mean amplitude, wave-form/timbre, mean sound frequency, polarization of single pulses, total duration of stimulation, duty-cycle;
e) setting said Mel-PARᵢₙ parameters in said software (S) provided in said processor (P), and transfer said Mel-PARᵢₙ parameters to said processor (P);
f) subjecting said subject to an initial stimulation cycle defined by said parameters Mel-PARᵢₙ;
g) during or at the end of said initial cycle, analyzing the expression melatonin level in said subject in order to obtain a Mel1 value;
h) storing said Mel_{TG}, Mel_{IN}, Mel1 and Mel-PARᵢₙ values into said memory unit (M) associated with said processor (P), and add Mel-PARᵢₙ to said PAR set;
i) optionally, perform the following sub-steps:
select Mel-PARᵢ₋₁ values from said PAR set wherein i is an integer with 2≤i≤n, said values being equal to or different from Mel-PARᵢ₋₂ or, alternatively, change one or more of said stimulation parameters Mel-PARi−2 in order to define new Mel-PARᵢ₋₁ values, wherein i is an integer with 2≤i≤n and Mel-PARin=Mel-PAR0, said stimulation parameters Mel-PARᵢ₋₁ being changed as follows:
the total stimulation duration of a value ranging from 0.1 to 10 minutes for each picograms per milliliter of melatonin concentration Melᵢ in said subject; and/or
the mean wavelength of the single pulses of a value ranging from 1 nm to 100 nm for each picograms per milliliter melatonin concentration Melᵢ in said subject; and/or
the mean sound frequency of the single pulses of a value ranging from 0.1 to 200 Hz for each picograms per milliliter of melatonin concentration Melᵢ in said subject; and/or
the photostimulation intensity of a time period between 0.1 and 10 microwatt for each picograms per milliliter of melatonin concentration Melᵢ in said subject;
set said Mel-PARᵢ₋₁ parameters in said software (S) provided in said processor (P), and transfer to said processor (P) said parameters Mel-PARᵢ₋₁;
using said parameters Mel-PARᵢ₋₁, perform on said subject an additional stimulation cycle, wherein said stimulation is selected from: photostimulation by visible or invisible light pulses, phonostimulation by audible or non-audible acoustic pulses, or a combination thereof;
during or at the end of the i-cycle, wherein i is an integer with 2≤i≤n, analyze the melatonin expression level in said subject so as to obtain a Melᵢ value associated to Mel-PARᵢ₋₁ values of said stimulation parameters;
store said Melᵢ and Mel-PARᵢ₋₁ values in said memory unit (M) associated with said processor (P),
optionally, repeat step i) for a number n of cycles, wherein n is an integer with n≤2 until said Melᵢ value reaches a Melₙ=Mel_{FIN}, value, said Melₙ value being substantially equal to Mel_{TG} or substantially constant, said Mel_{TG} value corresponding to the optimum melatonin expression value for said subject, said value being associated to a set Mel-PARₙ₋₁ of optimal stimulation parameters for the subject;
store in said memory unit (M) said final value Melₙ and said parameters set Mel-PARₙ₋₁ and add Mel-PARₙ₋₁ to said PAR set;
j) optionally, perform on said subject one or more periodic stimulation cycles by setting in said device the optimum stimulation parameters stored in step h) or i).

10. The method according to claim 9, further comprising a step wherein the cerebral waves of said subject are recorded and the melatonin expression level data of said subject are compared with the features of said cerebral waves.

11. The method according to claim 9 for the treatment of pathologies associated with oxidative stress or melatonin deficiency in a human or a mammal, said diseases and conditions being selected from the group consisting of: pigmentous retinitis, maculopathy, ocular disorders and diseases induced by extracellular protein accumulation, diabetes, tinnitus, dizziness, insomnia, alopecia, treatment and rehabilitation of subjects affected by stroke, and combinations thereof.

12. Use of the device according to claim 1 for the treatment of pathologies associated with oxidative stress or melatonin deficiency in a human or a mammal, said diseases being selected from the group consisting of: pigmentous retinitis, maculopathy, ocular disorders and diseases induced by extracellular protein accumulation, diabetes, tinnitus, dizziness, insomnia, alopecia, treatment and rehabilitation of subjects affected by stroke, or a combination thereof.

13. Use of the method according to claim 9 for increasing athletes performances in sport activities.
